(19) United States
(12) Patent Application Publication
(10) Pub. No.: US 2008/0194471 A1
(43) Pub. Date: Aug. 14, 2008

(54) Compositions and Methods for Treating Wounds

(75) Inventor: Robert S. Cutler, Jupiter, FL (US)

Correspondence Address: SYNNESTVEDT & LECHNER, LLP, 1101 MARKET STREET, SUITE 2600, PHILADELPHIA, PA, 19107-2950, US

(73) Assignee: OTKRYTOE AKTSYONERNOE OBSHCHESTVO "POLEMA", Tula (RU)

(21) Appl. No.: 12/063,548

(22) PCT Filed: Aug. 10, 2006
(86) PCT No.: PCT/US06/31317

§ 371 Date: Feb. 11, 2008

Related U.S. Application Data
(60) Provisional application No. 60/707,189, filed on Aug. 10, 2005.

(51) Int. Cl. A61K 38/12; A61P 17/00

(52) U.S. Cl. 514/11

(57) ABSTRACT

Disclosed are compositions for the treatment of anorectal disorders comprising at least one analgesic, at least one anti-inflammatory agent, antibiotic, and optionally but preferably hyaluronic acid, in amounts and under conditions effective to treat said anorectal disorder.

### FIELD OF THE INVENTION

This present invention relates to compositions and methods for treating wounds in general, and in particular applications to the treatment of anorectal disorders.

### BACKGROUND OF THE INVENTION

Conditions known as anal fissure (fissure-in-ano), anal ulcer, hemorrhoidal diseases, and levator spasm (proctalgia fugax) are, in general, relatively common benign conditions of the anorectal area which affect subjects, including humans, of all ages, races, and sexes. Although hemorrhoids and anal fissures typically do not receive the attention given to life threatening diseases, they are frequently responsible for considerable suffering, disability and/or embarrassment. Millions of people worldwide are affected by such unfortunate conditions.

In general, an anal fissure or ulcer is a tear, rip or the like of the mucosa or lining tissue at or near the opening of the anal canal. In some cases an anal fissure or ulcer may be associated with another systemic or local disease. An individual with an anal fissure or ulcer frequently experiences anal pain and bleeding, the pain being more pronounced during and after bowel movements.

Hemorrhoids are specialized vascular areas lying subjacent to the anal mucosa. Symptomatic hemorrhoidal diseases are manifested by bleeding, thrombosis and/or prolapse of the hemorrhoidal tissues. Commonly, internal hemorrhoidal tissue bulges into the anal canal during defecation and results in bleeding and pain. As the tissue enlarges, further bleeding, pain, prolapse and thrombosis can ensue. The thrombosis of hemorrhoids is yet another cause of bleeding and pain.

Hemorrhoids are the most prevalent anorectal disorder and are the most common cause of hematochezia (i.e., passage of bloody stools). Treatment is typically dependent on the degree of hemorrhoid prolapse and symptoms. Most cases (first- and second-degree hemorrhoids) generally respond to conservative medical treatment (e.g., dietary changes, sitz baths) or non-surgical procedures (e.g., rubber band ligation). Acutely thrombosed external hemorrhoids are usually characterized by severe anal pain, and heretofore surgical excision of symptomatic thrombosed external hemorrhoids was commonly indicated within 48 to 72 hours of the onset of pain. Post-hemorrhoidectomy pain is typically severe, disproportionate to the surgery itself. In some cases narcotic analgesics have been used in a post-surgical treatment, but such use can unfortunately complicate recovery by causing constipation.

Anal fissure is one of the most common causes of anorectal pain. Anal fissures are tears in the mucosa of the distal anal canal, usually along the posterior midline. The exact causes of anal fissures remain unknown. They are often associated with trauma, e.g., passage of a hard stool, but can also occur during bouts of diarrhea, childbirth, or ulceration of a hemorrhoid. The most common symptom is pain at defecation, which can be quite severe and last for a variable time afterwards. Many acute anal fissures will heal within three weeks using a known treatment regimen comprising sitz baths, stool softeners, and analgesics. However, it is not uncommon that acute anal fissures do not heal as a result of such heretofore used treatments, and in such cases it is possible that chronic anal fissures or anal ulcers are produced. Chronic anal fissures have heretofore not been typically responsive to conservative medical therapy, and therefore current treatments are frequently directed at surgical techniques, including anal dilatation (under anesthesia), or more commonly, lateral sphincterotomy of the internal anal sphincter. While healing occurs following surgical sphincterotomy in many cases, successful sphincterotomy (or anal dilatation) is associated with a significant decrease in intra-anal pressure and increase in anodermal blood flow, and as a result a large portion of patients may experience some form of pain and/or incontinence following the surgical procedure.

Applicant has therefore recognized a largely unmet medical need for effective, non-surgical treatments wound healing and/o treatment of maladies of the skin or flesh, including particularly for treatment anal fissure and other colorectal conditions, including anal fissures, acute hemorrhoidal disease, and like conditions.

### SUMMARY OF THE INVENTION

One aspect of the present invention provides compositions for the treatment of wounds in general and anorectal disorders in particular. The preferred compositions comprising at least one antibiotic, at least one analgesic, at least one anti-inflammatory agent, and optionally but preferably hyaluronic acid, in amounts and under conditions effective to treat said anorectal disorder.

The present invention further provides methods of using these compositions. Preferred methods of the invention comprise administering to a subject a suitable formulation of the present composition. In related methods, treatment is carried out by administration to the area affected of two or more of the components mentioned above in connection with the compositions in sequence, either by the same route of administration or by different routes of administration.

Applicants have discovered compositions which possess a highly desirable and unexpectedly superior combination of properties, particularly in connection with treatment of anorectal disorders and similar disorders. In preferred embodiments, the compositions and methods possess properties that have heretofore not been associated with topical treatment regimes.

Definitions

The terms “treatment”, “therapy” and the like include, but are not limited to, changes in the recipient's status. The changes can be either subjective or objective and can relate to features such as symptoms or signs of the disease or condition being treated. For example, if the patient notes decreased itching, reduced bleeding, reduced discomfort or decreased pain, then successful treatment has occurred. Similarly, if the clinician notes objective changes, such as by histological analysis of a biopsy sample, then treatment has also been successful. Alternatively, the clinician may note a decrease in the size of lesions or other abnormalities upon examination of the patient. This would also represent an improvement or a successful treatment. Preventing the deterioration of a recipient's status is also included by the term. Therapeutic benefit includes any of a number of subjective or objective factors indicating a response of the condition being treated as discussed herein.

“Drug”, “pharmacological agent”, “pharmaceutical agent”, “active agent”, “agent” and the like are used interchangeably and are intended to have their broadest interpretation as to any therapeutically active substance which is delivered to a living organism to produce a desired, usually beneficial effect.

“Pharmaceutically-acceptable” or “therapeutically-acceptable” refers to a substance which does not substantially interfere with the effectiveness or the biological activity of the active ingredients and which is not substantially toxic to the host, which may be either human or animal, to which it is administered. “Therapeutically-effective amount” refers to the amount of an active agent sufficient to induce a desired biological result. That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

The term “anorectal area” is defined herein to include both the anus and the rectum region of a mammal. More particularly, the term includes the internal anal canal, the external anus and the lower rectum.

The term “subject” as used herein includes any animal, such as a mammal, including a human.

The term “anorectal disorder” includes any disorder associated with an anal rectal disease, including an acute or chronic anal fissure, an internally or externally thrombosed hemorrhoid, a hemorrhoidal disease, a disorder associated with endoscopic hemorrhoidal ligation or pain caused by such ligation, and like conditions. The term also refers to post-surgical pain associated with hemorrhoidectomy or other anorectal surgery. Additionally, the term is meant to include pain which can be associated with any of the above disorders or conditions.

The term “desirable therapeutic effects” in the treatment of anorectal diseases and conditions includes, but is not limited to, reduction or elimination of ischemia, itching, inflammation, pain, bleeding, partial or complete healing of linear or ischemic ulcers or crack-like sores in the anal canal or on the margin of the anus, and the like.

The term “pharmaceutical composition” means a composition suitable for pharmaceutical use in a subject, including an animal or human. A pharmaceutical composition generally comprises an effective amount of an active agent and a pharmaceutically acceptable carrier.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in REMINGTON'S PHARMACEUTICAL SCIENCES (Mack Publishing Co., Easton, 19th ed. 1995), which is incorporated herein by reference. Preferred pharmaceutical carriers depend upon the intended mode of administration of the active agent. Typical modes of administration are described hereinafter.

The term “effective amount” means a dosage sufficient to produce a desired result. The desired result may comprise a subjective or objective improvement in the recipient of the dosage.

A “therapeutic treatment” is a treatment administered to a subject who exhibits signs of pathology, wherein treatment is administered for the purpose of diminishing or eliminating those pathological signs.

The term “appropriate anal area” means any area or tissue of the anus or sphincter that is affected by or subject to anal disorder or disease, including, for example, the external or internal anus, external or internal anal canal.

The terms “wound” and “wound” mean an undesirable condition of the flesh or skin, which in many cases is associated with discomfort or pain.

### DETAILED DESCRIPTION OF PREFERRED EMBODIMENTS

In general, this invention relates to the treatment of wounds, disease and conditions of the skin, flesh and/or exposed tissue. The present inventor has developed compositions that help to satisfy the continuing need for effective treatment of such conditions, including wound healing compositions and methods, particularly and preferably embodiments wherein the condition or wound comprises tears, fissures and the like, and particularly such conditions existing in the anal area. One aspect of the invention thus pertains to compositions comprising a synergistic combination of agents to produce results not heretofore achieved and/or contemplated. In preferred embodiments the present compositions comprise a therapeutically effective amount of antibacterial agent, a therapeutically effective amount of analgesic agent, and a therapeutically effective amount of anti-inflammatory agent. In preferred but optional embodiments, the compositions further comprise a transport agent for the other compositions to aid and enhance delivery and effective transport of the other components to the site of the pathology, condition, wound, fissure, tear, and the like. In certain preferred embodiments, the compositions comprise an effective non-toxic dosage amount of a form of hyaluronic acid (preferably hyaluronic acid or salt thereof) which preferably serves the purpose of aiding transport of the drug to the site of the pathology and/or trauma.

The amount of antibacterial agent in the present compositions is preferably a therapeutically effective amount. The exact amount of antibacterial agent may vary within a wide range and may be adjusted in view of the teachings contained herein for any particular case depending upon several factors, including the condition being treated as well as the other ingredients in the composition. In certain preferred embodiments, antibacterial agent is present in the composition in an amount of from about 0.01% to about 99%, preferably from about 50% to about 98%, and more preferably from about 75% to about 95%, by weight of the composition, based on the total of the antibacterial agent, the analgesic agent, the anti-inflammatory agent, and the transport agent. In preferred embodiments the above noted percentages are based on antibacterial agent in the form of bacitracin (preferably at least about 400 gm per unit, or in some cases preferably about 500 gm per unit). In an alternative embodiment the antibacterial agent is in the form of a combination of bacitracin (preferably from about 400 to about 500 gm per unit), neomycin (preferably 3.5 mg/gm) and polymyxin (preferably 10,000 units per gm). In yet a further embodiment the antibacterial agent is in the form of 2% mupirocin.

The amount of analgesic agent in the present compositions is preferably a therapeutically effective amount. The exact amount of analgesic agent may vary within a wide range and may be adjusted in view of the teachings contained herein for any particular case depending upon several factors, including the condition being treated as well as the other ingredients in the composition. In certain preferred embodiments, the analgesic agent is present in the composition in an amount of from about 0.2% to about 10%, preferably from about 0.5% to about 8%, and more preferably from about 0.5% to about 5%, by weight of the composition, based on the total of the antibacterial agent, the analgesic agent, the anti-inflammatory agent, and the transport agent. In preferred embodiments the above noted percentages are based on 4% lidocaine. In an alternative embodiment the antibacterial agent is in the form of 1% pramoxine hydrochloride.

The amount of anti-inflammatory agent in the present compositions is preferably a therapeutically effective amount. The exact amount of anti-inflammatory agent may vary within a wide range and may be adjusted in view of the teachings contained herein for any particular case depending upon several factors, including the condition being treated as well as the other ingredients in the composition. In certain preferred embodiments, the anti-inflammatory agent is present in the composition in an amount of from about 0.1% to about 5%, more preferably from about 0.5% to about 2.5%, by weight of the composition, based on the total of the antibacterial agent, the analgesic agent, the anti-inflammatory agent, and the transport agent. In preferred embodiments the above noted percentages are based on 1% USP hydrocortisone.

The amount of transport agent in the present compositions is preferably a therapeutically effective amount. The exact amount of transport agent may vary within a wide range and may be adjusted in view of the teachings contained herein for any particular case depending upon several factors, including the condition being treated as well as the other ingredients in the composition. In certain preferred embodiments, the transport agent is present in the composition in an amount of from about 0% to about 5%, more preferably from about 0.1% to about 2.5%, by weight of the composition, based on the total of the antibacterial agent, the analgesic agent, the anti-inflammatory agent, and the transport agent. In preferred embodiments the above noted percentages are based on 0.2% hyaluronic acid sodum salt. Without necessarily being bound or to any particular theory of operation, it is believed that the hylauronic acid form may enhance immune cell acitivation and/or tissue hydration and/or proteoglycan organization, in addition to helping to distribute the other ingredients of the composition on the skin or tissue being treated. In any event, it is believed that the hylauronic acid form of the present invention acts in a synergistic manner with the other ingredients to greatly enhance would healing and/or symptom relief.

The present invention extends to methods of making the therapeutic wound healing compositions of the present invention. In general, the present compositions are made by forming an admixture of the components of the composition.

One aspect of the present invention extends to methods for using the present compositions. In general, the present compositions are used by contacting the subject with the therapeutic composition, preferably at or near the anorectal area. Preferably the composition is used in such treatments in association with a pharmaceutically acceptable carrier. For example, in certain embodiments the present compositions comprise and may be applied in the form of anorectal creams and suppositories to treat such conditions as pruritus, and proctitis, anal fissures, and hemorrhoids. When used as a cream or ointment, it is preferred to include the present compositions in a hydrophilic ointment or petroleum base. In other embodiments, a cream base, a gel base or a foam base may be used. In other embodiments, an insertion suppository may be used, which in preferred embodiments comprises topical starch (about 50%) and inactive ingredients such as benzyl alcohol, hydrogenated vegetable oil, and tocopheryl acetate. When used in the form of or as part of a suppository, it is believed that the present compositions, additional advantage may be achieved by allowing the formation of an additional protective layer or barrier over the tissue or skin being treated.

Suitable dosage amounts of the composition may be removed from a container (for example a tube or jar) and administered (for example, applied using a cotton swab, gloved finger or the like). The particular amount and period of application will be known to those skilled in the art in view of the teachings contained herein.

Accordingly the combination of ingredients set out in the above embodiments function together in an enhanced manner to prevent and/or reduce injury to mammalian cells, and/or increase the resuscitation rate of injured mammalian cells, and/or cause the proliferation of new cells and/or relief the pain associated with the would and/or wound healing. The therapeutic effects of the combination of the components in each of the above embodiments is markedly greater than that expected by the mere addition of the individual therapeutic components.

What is claimed is:

1. A composition for the treatment of wounds in animal skin or flesh comprising at least one antibiotic, at least one analgesic, at least one anti-inflammatory agent, said components being present in the composition in amounts and under conditions effective to aid the healing of said wounds.

2. The composition of claim 1 further comprising a transport agent.

3. The composition of claim 1 further comprising hyaluronic acid or a salt thereof.

4. The composition of claim 1 wherein said antibacterial agent is present in the composition in an amount of from about 75% to about 95% by weight of the total of the antibacterial agent, the analgesic agent and the anti-inflammatory agent.

5. The composition of claim 1 wherein said antibacterial agent comprises bacitracin.

6. The composition of claim 1 wherein said antibacterial agent comprises bacitracin and neomycin.

7. The composition of claim 1 wherein said antibacterial agent comprises bacitracin, neomycin and polymyxin.

8. The composition of claim 1 wherein said analgesic agent is present in the composition in an amount of from about 0.2% to about 10% by weight of the total of the antibacterial agent, the analgesic agent and the anti-inflammatory agent.

9. The composition of claim 1 wherein said analgesic agent comprises lidocaine.

10. The composition of claim 1 wherein said analgesic agent comprises pramoxine hydrochloride.

11. The composition of claim 1 wherein said anti-inflammatory agent is present in the composition in an amount of from about 0.1% to about 0.5% by weight of the total of the antibacterial agent, the analgesic agent and the anti-inflammatory agent.

12. The composition of claim 1 wherein said anti-inflammatory agent comprises hydrocortisone.

13. A dose of medicament comprising the composition of claim 1 wherein said antibacterial agent is present in an amount of at least about 400 gm per unit.

14. A composition for the treatment of anorectal disorders in animal skin or flesh comprising at least one antibiotic, at least one analgesic, at least one anti-inflammatory agent and hyaluronic acid, said components being present in the composition in amounts and under conditions effective to aid the healing of said disorder.

15. A method for the treatment of a wound in animal skin or flesh comprising contacting the subject with a therapeutic composition comprising at least one antibiotic, at least one analgesic, and at least one anti-inflammatory agent, said contacting step being carried out under conditions effective to aid the healing of said wounds.

16. The method of claim 15 wherein said wound is in the anorectal area.

17. The method of claim 16 wherein said composition is in applied to the subject by contacting the subject with an anorectal cream.

18. The method of claim 16 wherein said composition is in applied to the subject by contacting the subject with a suppository.

19. The method of claim 15 wherein said wound comprises pruritus.

20. The method of claim 15 wherein said wound comprises proctitis.

21. The method of claim 15 wherein said wound comprises anal fissures.

22. The method of claim 15 wherein said wound comprises hemorrhoids.
